(19) United States
(12) Patent Application Publication
(10) Pub. No.: US 2024/0419020 A1
(43) Pub. Date: Dec. 19, 2024

(54) ATOMIZATION GLASSES

(71) Applicant: SUZHOU 3N BIOLOGICAL TECHNOLOGY CO., LTD., Suzhou (CN)

(72) Inventors: Bixia SUN, Suzhou (CN); Shengjiang XU, Suzhou (CN); Lei JIA, Suzhou (CN)

(73) Assignee: SUZHOU 3N BIOLOGICAL TECHNOLOGY CO., LTD., Suzhou (CN)

(21) Appl. No.: 18/813,717

(22) Filed: Aug. 23, 2024

Related U.S. Application Data
(63) Continuation of application No. PCT/CN2023/078331, filed on Feb. 27, 2023.

(30) Foreign Application Priority Data
Feb. 28, 2022 (CN) 202210187555.6

(51) Int. Cl. G02C 11/00

(52) U.S. Cl. CPC G02C 11/10

(57) ABSTRACT

The present disclosure discloses atomization glasses including a glasses frame, and a left lens, a right lens, and an atomization water tank that are respectively arranged on the glasses frame. The atomization water tank is located between the left lens and the right lens. An accommodating cavity is provided in the atomization water tank. An atomization hole is provided on at least one side of the atomization water tank. The atomization hole is in communication with the accommodating cavity. An atomization sheet is arranged at the atomization hole. A power supply assembly is arranged on the glasses frame. The atomization sheet is electrically connected to the power supply assembly. According to this application, the atomization water tank can be conveniently carried and replaced or cleaned by a user.

### CROSS-REFERENCE TO RELATED APPLICATIONS

This application is a continuation of International Patent Application PCT/CN2023/078331 filed Feb. 27, 2023, which claims priority benefit to Chinese Patent Application CN 202210187555.6 filed Feb. 28, 2020. The content of the above-mentioned applications is incorporated by reference in their entirety.

### TECHNICAL FIELD

The present disclosure relates to the technical field of glasses, and in particular, to atomization glasses.

### BACKGROUND

With the improvement of people's living standards and changes in the way people work, there has been an increasing demand for eye use. Moreover, with the advent of electronic products such as computers, mobile phones, and televisions, people often excessively and frequently use their eyes, resulting in an increasing number of patients with a dry eye syndrome. On the other hand, as people age, tears secreted by the eyes also decrease, which may also lead to the dry eye syndrome.

When the patient with the dry eye syndrome experience dryness and discomfort in their eyes, most people use artificial tears (eyedrops). But this can only temporarily relieve the discomfort and sometimes cause an increased frequency of eye dryness. Therefore, the artificial tears cannot effectively and completely relieve the dry eye syndrome.

### SUMMARY

Therefore, given the challenges in existing solutions, there is a need for new technical solutions.

The present disclosure provides atomization glasses that use an atomization sheet to atomize an eye moisturizing solution or similar liquid, thereby moisturizing the wearer's eyes.

To achieve the purpose of the present disclosure, the present disclosure provides atomization glasses, including a glasses frame, and a left lens, a right lens, and an atomization water tank that are respectively arranged on the glasses frame. The atomization water tank is located between the left lens and the right lens. An accommodating cavity is provided in the atomization water tank. An atomization hole is provided on at least one side of the atomization water tank. The atomization hole is in communication with the accommodating cavity. An atomization sheet is arranged at the atomization hole. A power supply assembly is arranged on the glasses frame. The atomization sheet is electrically connected to the power supply assembly.

Further, the glasses frame comprises a glasses frame body and a left temple and a right temple that are respectively arranged on two ends of the glasses frame body. The left lens, the right lens, and the atomization water tank are respectively arranged on the glasses frame body.

Further, the atomization water tank is arranged on a side of the glasses frame body in an extending direction of a free end of a temple.

Further, the power supply assembly comprises a first circuit board, a second circuit board is arranged in the atomization water tank, the atomization sheet is electrically connected to the second circuit board, and the first circuit board is connected to the second circuit board when the atomization water tank is arranged on the glasses frame body.

Further, the atomization water tank is detachably connected to the glasses frame body. The power supply assembly comprises a first conductive element and a second conductive element. The first conductive element and the second conductive element are respectively electrically connected to the first circuit board. A third conductive element and a fourth conductive element are arranged on the atomization water tank. The third conductive element and the fourth conductive element are respectively electrically connected to the second circuit board. When the atomization water tank is arranged on the glasses frame body, the first conductive element is connected to the third conductive element, and the second conductive element is connected to the fourth conductive element.

Further, a water level measuring element is further arranged on the atomization water tank. The water level measuring element is configured to measure an amount of solution in the atomization water tank, and the water level measuring element is connected to the first circuit board or the second circuit board.

Further, a nose pad structure is arranged on a side of the atomization water tank away from the glasses frame body.

Further, the power supply assembly comprises a third circuit board and a button member. The third circuit board is arranged in the left temple or the right temple, the third circuit board is electrically connected to the first circuit board, the button member is arranged on the glasses frame, and the button member is electrically connected to the third circuit board.

Further, the power supply assembly comprises a power storage member. The power storage member is arranged in the left temple or the right temple, and the power storage member is electrically connected to the third circuit board.

Further, an angle between a normal line of the atomization sheet and a middle glasses frame surface is in a range of 67° to 79°.

Further, the atomization glasses further comprise: a blink detection sensor, configured to obtain blink information of a user in real time; and a controller, respectively connected to the blink detection sensor and the atomization sheet, where the controller is configured to: determine a blink frequency value of a user within a set time period based on the blink information, compare the blink frequency value with each of a number of preset blink frequency thresholds to determine a blink frequency range where the user is currently located, and then control an operating duration or an atomization amount of the atomization sheet based on a control parameter corresponding to the determined blink frequency range.

Further, the blink detection sensor is a distance sensor, a photoelectric sensor, or an eye potential sensor.

The atomization glasses of this application have at least one or more of the following beneficial effects.

The atomization glasses of this application are designed with an independent detachable atomization water tank, so that the atomization water tank can be conveniently carried and replaced or cleaned by a user. The atomization sheet is arranged on the atomization water tank, so as to atomize the atomizable liquid such as eye moisturizing solution in the atomization water tank, and spray the atomized atomizable liquid onto the eyeballs of the wearer to wet the eyeballs. The angle between the normal line of the atomization sheet and the middle glasses frame surface is designed to cover a range of the eyeballs. Dual protection is provided for the atomization sheet by arranging a water level measuring device in the atomization water tank to prevent dry heating of the atomization sheet and arranging a circuit to detect and prevent dry heating. A main circuit board and the power storage member are both arranged in the temple on the same side. The glasses are easy to assemble. The glasses may be put into a glasses case for charging through a charging port arranged at a temple.

In the atomization glasses of this application, the blink detection sensor may further be arranged to detect the blink information of the user in real time, and an eye state of the user is determined based on the blink frequency of the user, thereby intelligently adjusting and controlling the operating duration or the atomization amount of the atomization sheet.

### BRIEF DESCRIPTION OF THE DRAWINGS

FIG. 1 is a three-dimensional schematic structural diagram of atomization glasses according to one embodiment of this application;

FIG. 2 is a schematic diagram of an explosion structure of atomization glasses according to one embodiment of this application;

FIG. 3 is a schematic diagram of an explosion structure of an atomization water tank according to one embodiment of this application;

FIG. 4 is a schematic cross-sectional view of an atomization water tank according to one embodiment of this application;

FIG. 5 is a schematic cross-sectional view of an atomization sheet according to one embodiment of this application;

FIG. 6 is a schematic diagram of an angle between a normal line of an atomization sheet and a middle glasses frame surface according to one embodiment of this application;

FIG. 7 is a schematic exploded view among atomization glasses, a glasses case, a liquid medicine bottle, and a spare atomization water tank according to one embodiment of this application;

FIG. 8 is a schematic diagram of an explosion structure of a glasses case according to one embodiment of this application; and

FIG. 9 is a flowchart of controlling an atomization sheet according to one embodiment of this application.

1—Glasses frame, 11—Glasses frame body, 111—Cover plate, 112—Baffle, 113—Lens stand, 114—Body base, 12—Left temple, 13—Right temple, 131—Temple body, 132—Temple cover plate, 133—Temple baffle, 134—Light guide post, 135—Charging plate, 14—First circuit board, 15—First conductive element, 16—Second conductive element, 17—Third circuit board, 18—Button member, 19—Power storage member, 2—Left lens, 3—Right lens, 4—Atomization water tank, 41—Water tank body, 411—Atomization hole, 412—Accommodating groove, 413—Atomization sheet accommodating cavity, 414—Nose pad structure, 415—Liquid adding hole, 416—Bump, 42—Second circuit board, 43—Atomization sheet, 431—Sealing member, 44—Water tank side cover, 45—Third conductive element, 46—Fourth conductive element, 47—Water level measuring element, 48—Sealing cover, 5—Nose pad, 6—Normal line of the atomization sheet, 7—Middle glasses frame surface, 8—Glasses case, 81—Case body, 811—Case body housing, 8111—Bottom pad, 812—Middle case body cover, 8121—Receiving groove, 8122—Atomizable liquid receiving groove, 8123—Water tank receiving groove, 82—Case cover, 83—Circuit board, 84—Sixth conductive element, 85—Power storage device, 86—Magnet, 87—Elastic pin, 88—Power button, 89—Button circuit board, 891—Indicator light, 90—LED light guide member, 91—LED light-blocking foam, 9—Liquid medicine bottle, 10—Spare atomization water tank.

### DETAILED DESCRIPTION OF THE PREFERRED EMBODIMENTS

In order to further describe technical means and effects adopted in the present disclosure to achieve the intended purposes of the present disclosure, specific implementations, structures, features, and effects thereof according to the present disclosure are described in detail below with reference to the accompanying drawings and preferred embodiments.

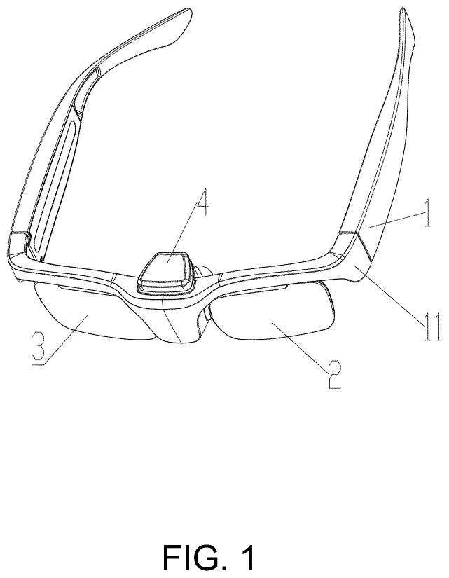

Atomization glasses are provided according to one embodiment. The atomization glasses are mainly composed of a glasses frame 1 and a left lens 2, a right lens 3, and an atomization water tank 4 that are respectively arranged on the glasses frame 1, as shown in FIG. 1. The glasses frame 1 comprises a glasses frame body 11 and a left temple 12 and a right temple 13 that are respectively arranged on two ends of the glasses frame body 11. The left lens 2, the right lens 3, and the atomization water tank 4 are respectively arranged on the glasses frame body 11.

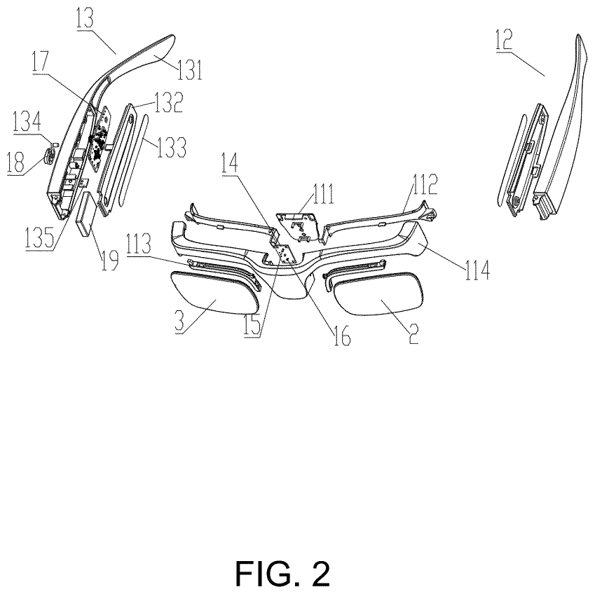

As shown in FIG. 2, a glasses frame 1 is schematically shown. The glasses frame body 11 is composed of a body base 114 and two baffles 112. The body base 114 is successively provided with a left lens fixing portion, a middle portion, and a right lens fixing portion along a length direction. The left lens fixing portion and the right lens fixing portion are each provided with an inner cavity, and are each provided with an opening on the same side. The opening is in communication with the corresponding inner cavity, and the two baffles 112 are respectively arranged at the corresponding openings to seal the inner cavities. An end portion of the left lens fixing portion and an end portion of the right lens fixing portion are respectively bent in an opening direction of the body base 114. That is, a fixing portion connected to the temple is formed on a side in an extending direction of a free end of the temple. The left temple 12 and the right temple 13 are respectively rotatably connected to the corresponding fixing portions. The middle portion extends downward, so that a region for the lenses to be arranged is formed between two sides of the middle portion and each of the left lens fixing portion and the right lens fixing portion. The left lens 2 and the right lens 3 are respectively arranged below the left lens fixing portion and the right lens fixing portion through a lens stand 113, that is, the two sides of the middle portion.

The middle portion is provided with a groove on a side of an opening direction of the glasses frame body 11. The atomization water tank 4 is detachably arranged in the groove, and is located between the left lens 2 and the right lens 3. A power supply assembly is arranged on the glasses frame 1, and is configured to supply power to the atomization water tank 4. The power supply assembly comprises a first circuit board 14, a first conductive element 15, and a second conductive element 16. The first conductive element 15 and the second conductive element 16 are respectively electrically connected to the first circuit board 14. Preferably, the first conductive element 15 and the second conductive element 16 are respectively arranged on the first circuit board 14. The first conductive element 15 and the second conductive element 16 may be conductive elements such as spring probes. The first circuit board 14 is arranged in the groove, and is a charging plate 135 of the atomization water tank 4. A side of the first circuit board 14 on which the first conductive element 15 and the second conductive element 16 are arranged is oriented outward. The groove is sealed by a cover plate 111. A hole is formed at a position of the cover plate 111 corresponding to each of the first conductive element 15 and the second conductive element 16. The cover plate 111 may be fixedly connected to the glasses frame body 11 through a fastener such as a screw. Preferably, a number of limiting shafts are arranged on a side of the cover plate 111 toward the glasses frame body 11. The first circuit board 14 is provided with a through hole at a position corresponding to each of the limiting shafts. After the cover plate 111 is engaged with the groove, the limiting shaft extends through the corresponding through hole on the first circuit board 14, thereby limiting the first circuit board 14 in the groove.

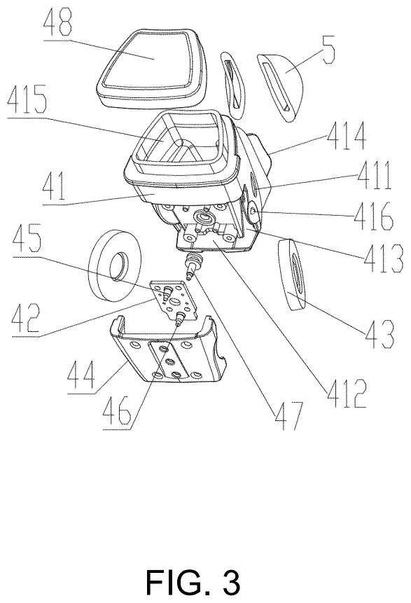

The atomization water tank 4 comprises a water tank body 41, a second circuit board 42, a third conductive element 45, a fourth conductive element 46, and a water tank side cover 44, as shown in FIG. 3. The third conductive element 45 and the fourth conductive element 46 are respectively electrically connected to the second circuit board 42. The water tank body 41 is preferably a box structure with an opening at an upper end, is made of a silver ion antibacterial plastic material, and is provided with an accommodating cavity therein for storing an atomizable liquid, and the opening thereof is a liquid adding hole 415. Certainly, the liquid adding hole 415 of the atomization water tank 4 may also be in another form. For example, a relatively small hole may be formed on the water tank body 41 as required. The hole is in communication with the accommodating cavity, and a liquid may also be added into the water tank body 41. To prevent the liquid in the water tank body 41 from overflowing during use, a sealing cover 48 may be arranged at the liquid adding hole 415 to seal the liquid adding hole 415. An accommodating groove 412 is arranged on a side of the water tank body 41 toward the glasses frame 1. The water tank side cover 44 is engaged with the accommodating groove 412, and the second circuit board 42 is arranged in the accommodating groove 412. The third conductive element 45 and the fourth conductive element 46 are preferably arranged on the second circuit board 42. The third conductive element 45 and the fourth conductive element 46 may be the conductive elements such as the spring probes. A side of the second circuit board 42 provided with the third conductive element 45 and the fourth conductive element 46 is oriented toward a water tank side plate. A hole is formed at a position of the water tank side plate corresponding to each of the third conductive element 45 and the fourth conductive element 46, so that the third conductive element 45 and the fourth conductive element 46 can be brought into communication with the outside. The water tank side plate may be fixedly connected to the water tank body 41 through the fastener such as the screw. Preferably, a number of limiting posts are arranged in the accommodating groove 412. The second circuit board 42 is respectively provided with a through hole at a position corresponding to each of the limiting posts, and the second circuit board 42 is limited and fixed through the number of limiting posts.

The atomization water tank 4 is preferably matched with the glasses frame 1 through a bump 416, to implement a detachable mounting manner. For example, a bump 416 is arranged on each of left and right sides of the atomization water tank 4. However, a recess or a small hole is provided at a position of each of inner walls of both sides of the groove of the middle portion corresponding to the bump 416. When the atomization water tank 4 is mounted to the groove, the bump 416 is engaged with the corresponding recess or small hole. Certainly, a detachable manner between the atomization water tank 4 and the glasses frame 1 is not limited thereto, other detachable structural forms may also be used. The third conductive element 45 and the fourth conductive element 46 are respectively arranged corresponding to the first conductive element 15 and the second conductive element 16. When the atomization water tank 4 is arranged on the glasses frame body 11, the first conductive element 15 is connected to the third conductive element 45, and the second conductive element 16 is connected to the third conductive element 45, thereby implementing an electrical connection between the first circuit board 14 and the second circuit board 42.

Further, a water level measuring element 47 may further be arranged on the atomization water tank 4, and is configured to measure an amount of solution in the atomization water tank 4, to prevent dry heating of an atomization sheet 43. As shown in FIG. 3, preferably, the water level measuring element 47 uses a water level measuring head arranged in the accommodating groove 412. An end of the water level measuring element 47 extends through a bottom of the accommodating groove 412 and is in communication with an inner cavity of the water tank body 41, and another end of the water level measuring element 47 extends through the second circuit board 42. Similarly, a hole is formed at a position of the water tank side plate corresponding to the water level measuring element 47, so that the water level measuring element 47 can be brought into communication with the outside. However, a fifth conductive element is arranged at a position of the first circuit board 14 corresponding to the water level measuring element 47. The fifth conductive element is connected to the first circuit board 14, and a hole is formed at a position of the cover plate 111 corresponding to the fifth conductive element. Therefore, when the atomization water tank 4 is arranged on the glasses frame body 11, the water level measuring element 47 is electrically connected to the fifth conductive element, that is, electrically connected to the first circuit board 14. Certainly, the water level measuring element 47 may also be directly connected to the second circuit board 42. Certainly, dual protection may also be provided for the atomization sheet 43 by arranging a circuit to detect and prevent dry heating.

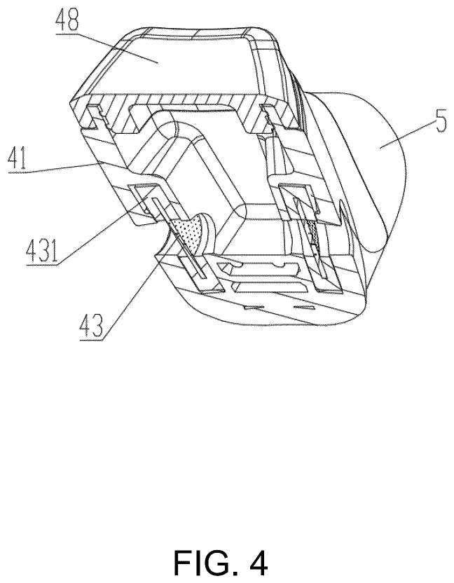

An inner wall of each of left and right sides of the water tank body 41 is provided with an atomization sheet accommodating cavity 413, and the atomization sheet accommodating cavity 413 is in communication with the accommodating groove 412, as shown in FIG. 4. Preferably, an atomization hole 411 is provided on the inner wall of each of the left and right sides of the water tank body 41, to implement an effect of simultaneously wetting left and right eyes. However, during specific implementation, the atomization hole 411 may also be provided on only one side as required. The atomization hole 411 extends through the atomization sheet accommodating cavity 413 and is in communication with an accommodating cavity of the water tank body 41. The atomization sheet 43 is arranged in the atomization sheet accommodating cavity 413, and the atomization sheet 43 is connected to the second circuit board 42.

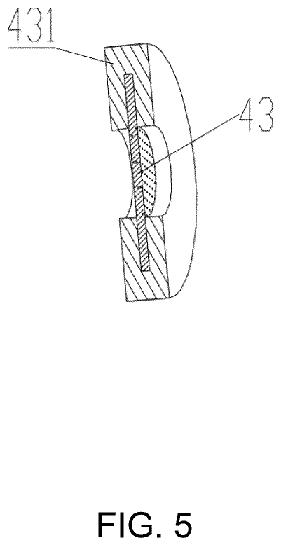

A sealing member 431 is sleeved on the atomization sheet 43, for example, a sealing silicone sleeve. A through hole is provided on the sealing member 431, as shown in FIG. 5. When the atomization sheet 43 is mounted to the atomization sheet accommodating cavity 413, the through hole is brought into communication with the atomization hole 411. The sealing member 431 is mainly configured to play a role of sealing and prevent the atomizable liquid in the water tank body 41 from leaking from the periphery of the atomization sheet 43.

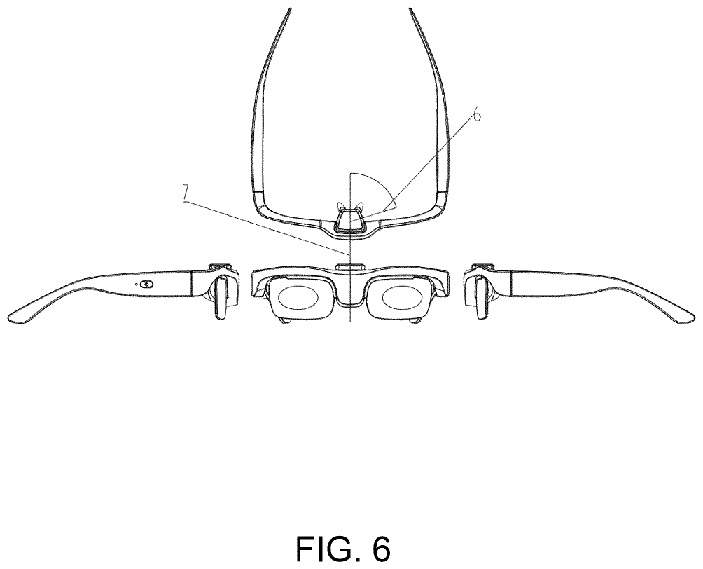

An angle between a normal line 6 of the atomization sheet and a middle glasses frame surface 7 is preferably in a range of 67° to 79°, as shown in FIG. 6. In this way, an eyeball region may be substantially covered.

Further, a nose pad structure 414 is further arranged at a position close to a bottom of a side of the water tank body 41 away from the glasses frame 1. A nose pad 5 may be arranged on the nose pad structure 414, so as to support the atomization glasses when a user uses the atomization glasses.

In a further embodiment, the power supply assembly further comprises a third circuit board 17, a button member 18, and a power storage member 19. The third circuit board 17 is arranged in the left temple 12 or the right temple 13. The third circuit board 17 is electrically connected to the first circuit board 14. The button member 18 is arranged on the glasses frame 1, and the button member 18 is electrically connected to the third circuit board 17. The power storage member 19 such as a battery is arranged in the left temple 12 or the right temple 13. The power storage member 19 is electrically connected to the third circuit board 17. Preferably, the third circuit board 17, the button member 18, and the power storage member 19 are arranged on the same temple, for example, the right temple 13, so that the assembling is more convenient. As shown in FIG. 2, a temple is schematically shown, which is mainly composed of a temple body 131 and a temple cover plate 132. An accommodating cavity is arranged in the temple body 131, and a side of the temple body 131 is provided with an opening. The temple cover plate 132 is arranged at the opening and seals the opening. The temple cover plate 132 and the temple body 131 are preferably fixedly connected through a fastener such as a screw. For aesthetics and reducing corrosion of the fastener, a temple baffle 133 is arranged on a side of the temple cover plate 132 away from the temple body 131, to shield the fastener. Moreover, for more convenience of the user to observe an operating state of the glasses, an indicator light may be arranged on the third circuit board 17. In addition, a light guide post 134 is arranged at a position of the temple body 131 corresponding to the indicator light, so that light emitted by the indicator light can be emitted smoothly and evenly.

An end portion of the temple body 131 is provided with a wire hole, so that when the temple is mounted to the glasses frame 1, the accommodating cavity in the temple body 131 may be brought into communication with an inner cavity of the body base 114 of the glasses frame body 11, and then a connecting line between the third circuit board 17 and the first circuit board 14 may be routed from inside.

For more convenience of carrying and charging, the atomization glasses in this embodiment are further provided with a glasses case 8. A power storage device 85 is arranged in the glasses case 8, so as to charge the atomization glasses when the atomization glasses are placed in the glasses case 8.

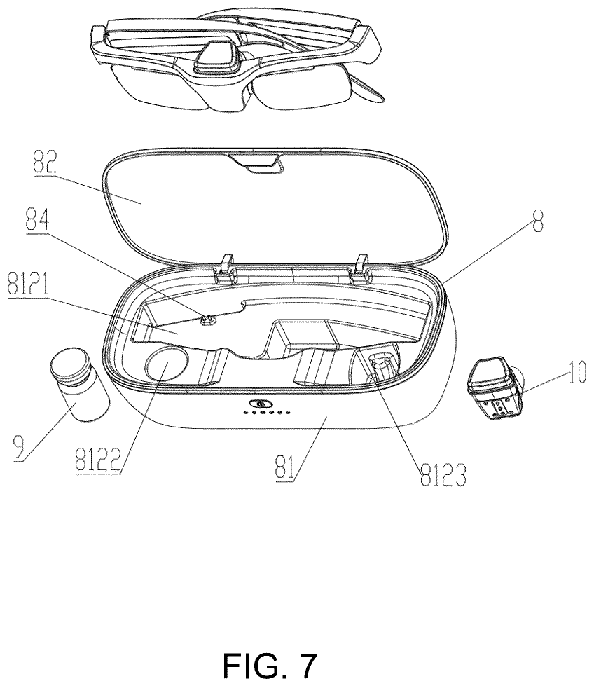

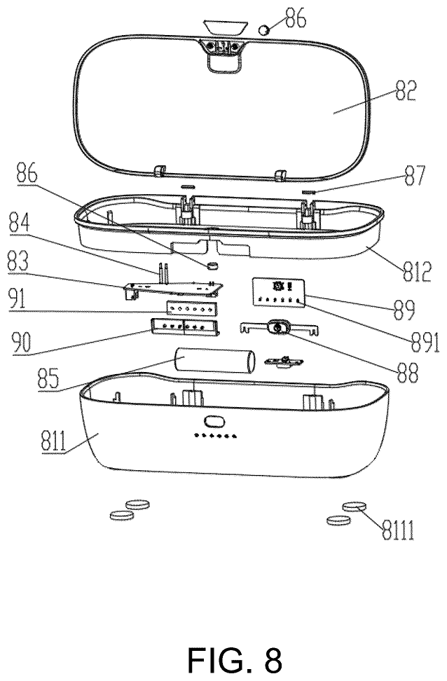

Specifically, as shown in FIG. 7 and FIG. 8, a glasses case 8 is schematically shown, which is mainly composed of a case body 81 and a case cover 82. An end of the case cover 82 is rotatably connected to the case body 81, and another end of the case cover 82 is connected to the case body 81 by magnetic adsorption through a magnet 86. The case body 81 is mainly composed of a case body housing 811 and a middle case body cover 812. The case body housing 811 has an inner cavity, and an upper end of the case body housing 811 has an opening. The opening is in communication with the inner cavity of the case body housing 811. The middle case body cover 812 is engaged at the opening of the case body housing 811. A side of the middle case body cover 812 away from the case body housing 811 is provided with a receiving groove 8121, and the atomization glasses are folded and placed in the receiving groove 8121. The case cover 82 is rotatably connected to the middle case body cover 812 through an elastic pin 87.

A circuit board 83 is arranged in the glasses case 8. The circuit board 83 and the power storage device 85 are respectively located between the case body housing 811 and the middle case body cover 812. The power storage device 85 is connected to the circuit board 83. A sixth conductive element 84 is arranged in the glasses case 8. An end of the sixth conductive element 84 is connected to the circuit board 83, and another end of the sixth conductive element 84 extends through the middle case body cover 812 into the receiving groove 8121. Correspondingly, a charging plate 135 corresponding to the sixth conductive element 84 is arranged on the atomization glasses. For example, as shown in FIG. 2, the charging plate 135 is also arranged in the temple, and the charging plate 135 is connected to the third circuit board 17. A small hole is formed at a corresponding position of the temple body 131, so that when the atomization glasses are placed in the glasses case 8, the sixth conductive element 84 can be electrically connected to the charging plate 135, and then the power storage device 85 can charge the atomization glasses.

Further, a power button 88 and a button circuit board 89 are further arranged on the glasses case 8. As shown in FIG. 8, the case body housing 811 is provided with a button hole on a side wall on an opening/closing end, and the power button 88 is arranged in the button hole. The button circuit board 89 is arranged in the case body housing 811 and is located on a side of the power button 88. The button circuit board 89 is connected to the circuit board 83. A switching element is arranged at a position of the button circuit board 89 corresponding to the power button 88.

Further, a number of indicator lights 891 are further arranged on the glasses case 8. As schematically shown in FIG. 8, the indicator lights 891 such as LED lights are arranged on the button circuit board 89 and are located below the switching element. Correspondingly, a number of light holes are formed on a side wall of the case body housing 811. To enhance an aesthetic appearance of a product and enable rays of light emitted by the indicator lights 891 to be emitted smoothly and evenly diffused, an LED light guide member 90 and an LED light-blocking foam 91 may further be arranged between the button circuit board 89 and the case body housing 811.

Further, an atomizable liquid receiving groove 8122 may be provided on a side of the middle case body cover 812 away from the case body housing 811, which is configured to store an atomizable liquid medicine bottle 9. A water tank receiving groove 8123 may further be provided on a side of the middle case body cover 812 away from the case body housing 811, which is configured to store a spare atomization water tank 10.

Further, bottom pads 8111 may further be arranged at a bottom of the case body housing 811 to provide anti-slip and support functions.

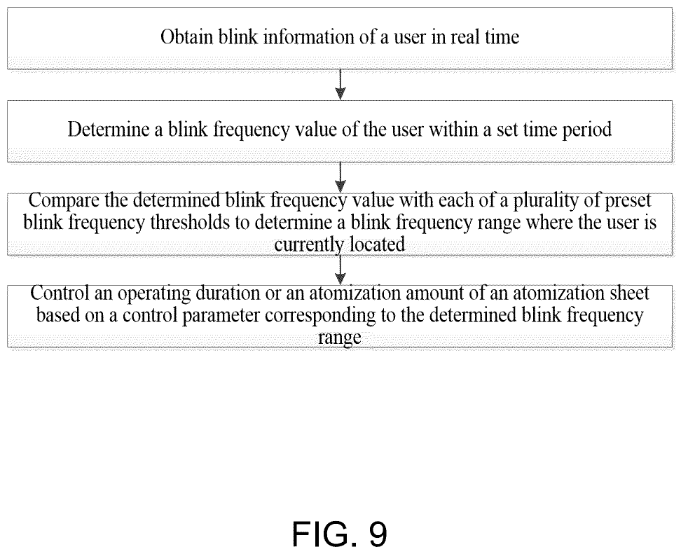

Further, a blink sensor and a controller may further be arranged on the atomization glasses to intelligently adjust and control an operating duration or an atomization amount of the atomization sheet 43. The controller may be a microcontroller unit (MCU) arranged on the third circuit board 17, for example, an atmega328p chip. A circuit of the third circuit board 17 is designed, so that the blink sensor and the atomization sheet 43 are respectively connected to the controller. A blink detection sensor is configured to obtain blink information of a user in real time. The blink detection sensor may be, for example, a photoelectric sensor such as a distance sensor or an infrared sensor, arranged on the glasses frame 1 and exactly facing an eyeball position. When the user blinks, the distance sensor or the photoelectric sensor detects a distance change and then transmits a detection signal to the controller, and the controller receives the signal and then determines that the user blinks once. The controller has a built-in algorithm, and calculates a blink frequency value of the user within a certain set time period based on a received blink signal, compare the blink frequency value with each of a number of preset blink frequency thresholds to determine a blink frequency range where the user is currently located, and control the operating duration or the atomization amount of the atomization sheet based on a control parameter corresponding to the determined blink frequency range. The control process is shown in FIG. 9. For example, a blink frequency per minute is preset to be greater than 40 times in the controller, and a control parameter of the controller for the atomization sheet is 100% power. When the blink frequency is less than or equal to 40 times or greater than 30 times, the control parameter is 50% power, or the like. When the controller calculates that the blink frequency of the user per minute is for example 35 times, the controller drives the atomization sheet to operate at 50% power, so as to wet the eyeballs of the user with a more appropriate atomization amount.

Certainly, the blink detection sensor is not limited to the aforementioned photoelectric sensor such as the distance sensor or the infrared sensor, it can also be other types of sensors, such as an electrooculography (EOG) sensor arranged at a corner of an eye near a temple. It may determine whether a user is blinking by detecting stable potential changes between a cornea and a retina.

The atomization glasses of this application have at least one or more of the following beneficial effects.

The atomization glasses of this application are designed with an independent detachable atomization water tank, so that the atomization water tank can be conveniently carried, replaced, or cleaned by a user. The atomization sheet is arranged on the atomization water tank, so as to atomize the atomizable liquid such as eye moisturizing solution in the atomization water tank, and spray the atomized atomizable liquid onto the eyeballs of the wearer to wet the eyeballs. The angle between the normal line of the atomization sheet and the middle glasses frame surface is designed to cover a range of the eyeballs. Dual protection is provided for the atomization sheet by arranging a water level measuring device in the atomization water tank to prevent dry heating of the atomization sheet and arranging a circuit to detect and prevent dry heating. A main circuit board and the power storage member are both arranged in the temple on the same side. The glasses are easy to assemble. The glasses may be put into a glasses case for charging through a charging port arranged at a temple.

In the atomization glasses of this application, the blink detection sensor may further be arranged to detect the blink information of the user in real time, and an eye state of the user is determined based on the blink frequency of the user, thereby intelligently adjusting and controlling the operating duration or the atomization amount of the atomization sheet.

Terms such as “comprise”, “include”, or any other variants thereof herein are intended to encompass a non-exclusive inclusion, meaning that in addition to the elements listed, other elements that are not explicitly listed may further be included.

Directional terms such as “front”, “back”, “up”, and “down” involved herein are defined by positions of parts in the accompanying drawings and between the parts, and are used solely for the clarity and convenience of explaining the technical solution. It should be understood that the use of the directional terms should not limit the protection scope of this application.

Where there is no conflict, the foregoing embodiments herein and features in the embodiments may be combined with each other.

The foregoing descriptions are merely preferred embodiments of the present disclosure, and are not intended to limit the present disclosure. Any modification, equivalent replacement, and improvement made without departing from the spirit and principle of the present disclosure shall fall within the protection scope of the present disclosure.

What is claimed is:

1. Atomization glasses, comprising a glasses frame, and a left lens, a right lens, and an atomization water tank that are respectively arranged on the glasses frame, wherein the atomization water tank is located between the left lens and the right lens, an accommodating cavity is provided in the atomization water tank, an atomization hole is provided on at least one side of the atomization water tank, the atomization hole is in communication with the accommodating cavity, an atomization sheet is arranged at the atomization hole, a power supply assembly is arranged on the glasses frame, and the atomization sheet is electrically connected to the power supply assembly.

2. The atomization glasses according to claim 1, wherein the glasses frame comprises a glasses frame body, and a left temple and a right temple that are respectively arranged on two ends of the glasses frame body, wherein the left lens, the right lens, and the atomization water tank are respectively arranged on the glasses frame body.

3. The atomization glasses according to claim 2, wherein the atomization water tank is arranged on a side of the glasses frame body in an extending direction of a free end of the temple.

4. The atomization glasses according to claim 3, wherein the power supply assembly comprises a first circuit board, a second circuit board is arranged in the atomization water tank, the atomization sheet is electrically connected to the second circuit board, and the first circuit board is connected to the second circuit board when the atomization water tank is arranged on the glasses frame body.

5. The atomization glasses according to claim 4, wherein the atomization water tank is detachably connected to the glasses frame body, the power supply assembly comprises a first conductive element and a second conductive element, the first conductive element and the second conductive element are respectively electrically connected to the first circuit board, a third conductive element and a fourth conductive element are arranged on the atomization water tank, the third conductive element and the fourth conductive element are respectively electrically connected to the second circuit board, and when the atomization water tank is arranged on the glasses frame body, the first conductive element is connected to the third conductive element, and the second conductive element is connected to the fourth conductive element.

6. The atomization glasses according to claim 5, wherein a water level measuring element is further arranged on the atomization water tank, the water level measuring element is configured to measure an amount of solution in the atomization water tank, and the water level measuring element is connected to the first circuit board or the second circuit board.

7. The atomization glasses according to claim 3, wherein a nose pad structure is arranged on a side of the atomization water tank away from the glasses frame body.

8. The atomization glasses according to claim 4, wherein the power supply assembly comprises a third circuit board and a button member, the third circuit board is arranged in the left temple or the right temple, the third circuit board is electrically connected to the first circuit board, the button member is arranged on the glasses frame, and the button member is electrically connected to the third circuit board.

9. The atomization glasses according to claim 8, wherein the power supply assembly comprises a power storage member, the power storage member is arranged in the left temple or the right temple, and the power storage member is electrically connected to the third circuit board.

10. The atomization glasses according to claim 1, wherein an angle between a normal line of the atomization sheet and a middle glasses frame surface is in a range of 67° to 79°.

11. The atomization glasses according to claim 1, further comprising:
a blink detection sensor, configured to obtain blink information of a user in real time; and
a controller connected to the blink detection sensor and the atomization sheet respectively, wherein the controller is configured to: determine a blink frequency value of a user within a set time period based on the blink information, compare the blink frequency value with each of a plurality of preset blink frequency thresholds to determine a blink frequency range where the user is currently located, and then control an operating duration or an atomization amount of the atomization sheet based on a control parameter corresponding to the determined blink frequency range.

12. The atomization glasses according to claim 11, wherein the blink detection sensor is a distance sensor, a photoelectric sensor, or an eye potential sensor.
